(19) United States
(12) Patent Application Publication
(10) Pub. No.: US 2011/0281006 A1
(43) Pub. Date: Nov. 17, 2011

(54) FLAVOR ENHANCING AGENT AND FLAVORING COMPOSITION

(75) Inventors: Yuji Nonaka, Kanagawa (JP); Mizuho Takahashi, Kanagawa (JP)

(73) Assignee: SUNTORY HOLDINGS LIMITED, Osaka-shi (JP)

(21) Appl. No.: 13/141,383

(22) PCT Filed: Dec. 25, 2009
(86) PCT No.: PCT/JP2009/071662

§ 371 Date: Aug. 3, 2011

(30) Foreign Application Priority Data
Dec. 26, 2008 (JP) 2008335192

(51) Int. Cl. A23L 1/221; A23L 2/00; A23F 3/16; C07D 407/10

(52) U.S. Cl. 426/536; 549/399

(57) ABSTRACT

There are provided a flavor enhancing agent which is capable of selectively enhancing the flavor of a flavoring of interest, as well as a flavoring composition and a drink that contain the agent and which are enhanced in flavor. Specifically, a polymerized catechin is used with a flavoring.

### TECHNICAL FIELD

The present invention relates to an agent for enhancing the flavor of foods and drinks, as well as a flavoring composition that has a flavoring enhanced in its flavor.

### BACKGROUND ART

The manufacture of foods and drinks involves heavy use of flavorings in order to increase the consumer's preference for them. However, flavorings are not generally high in stability and will diminish or disappear in the process of heat treatment such as sterilization and during storage, so efforts are being made to devise ways to enhance the stability of flavorings.

If foods and drinks are in a solid form, flavorings can be stabilized by sugar coating, use of coating agents, encapsulation, or use of inclusion compounds; however, if foods and drinks are in a liquid form (drinks), stable coating is more difficult to achieve than in the case of solid foods and drinks, so the method of using additives to enhance the flavor of flavorings is commonly adopted. For example, Patent Document 1 discloses a flavoring that has a free amino acid incorporated in it to be enhanced in flavor.

There have also been reported various methods to enhance the fragrance and taste of foods and drinks per se. For example, Patent Documents 2 and 3 disclose that when a taste enhancing agent comprising a tea extract with solvent and 3-galloylquinic acid or a salt thereof as active ingredients is added to a food or drink, palatability and body are imparted and/or enhanced to change the taste of the food or drink to a preferred one that is more complex and deeper; according to the documents, the technique was effective in consommé, coffee drinks, coffee hard candies, orange jelly, milk pudding, etc. Patent Document 4 describes a flavor enhancing agent comprising spirantol as an active ingredient, which may be added in a small amount to enhance the flavor of a food or drink per se without affecting the taste and fragrance of the food or drink.

Flavor enhancing agents for enhancing specific flavors have also been reported. For example, Patent Document 5 discloses that quinic acid derivatives can enhance fragrance and flavor, as exemplified by the refreshing sensation of menthol; Patent Document 6 discloses that a black tea extract improves the spicy sensation of curry or stew. Disclosures are also made about ways to enhance the characteristic flavor of fruit; Patent Document 7 describes an agent for enhancing the characteristic flavor of fruit, in which a liquid whey obtained by fermentation with a clarifying lactic acid bacterium or yeast is contained as an active ingredient; Patent Document 8 discloses that aspartic acid and/or a soluble salt thereof as well as alanine and/or threonine enhance the strawberry-like, grape-like, apple-like and melon-like flavors of foods and drinks; and Patent Document 9 discloses that threonine, lysine or a salt thereof, methionine, and valine enhance the pineapple-like flavor of foods and drinks.

### CITATION LIST

### Patent Documents

Patent Document 1: JP 2001-271089 A
Patent Document 2: JP 2005-137286 A
Patent Document 3: JP 2006-238815 A
Patent Document 4: JP 2006-296356 A
Patent Document 5: JP 2006-104229 A
Patent Document 6: JP 2006-34146 A (U.S. Pat. No. 4,105,130)
Patent Document 7: JP Hei 7-75521 A
Patent Document 8: JP Sho 60-227654 A
Patent Document 9: JP Hei 7-8205 B

### SUMMARY OF INVENTION

### Technical Problems

With the recent diversification of consumer's preference and increase in health consciousness, drinks of a new type have been developed; they are neutral drinks, such as water or tea beverages, with a certain flavor imparted thereto. There is, however, a problem with this product: flavorings, in particular fruit flavorings, usually give off their characteristic flavors (a fruit characteristic flavor in the case of a fruit flavoring) in drinks that contain an acidulant and a sweetener, but in the case of neutral drinks that have neither an acidulant nor a sweetener added thereto, they will not be effectively flavored by an added flavoring and the top note of the flavoring is extremely weak and the last note is also weak. A further problem is that if a flavoring is incorporated in a large quantity in a drink, a solvent of the flavoring will affect the flavor of the drink itself, and in the case of packaged drinks which are manufactured via a sterilization treatment, the balance of the flavoring will greatly change during the heat treatment or storage.

Another problem with the existing flavor enhancing agents is that the inherent flavors of foods and drinks that contain them are enhanced as such in their entirety, so in the case of a drink prepared by flavoring a tea-based beverage with a fruit flavoring, not only the flavor that is characteristic of the fruit but also the flavor of the tea which serves as the base of the drink is enhanced, making it impossible to ensure that only the flavor of the flavoring of interest is selectively enhanced.

An object of the present invention is to provide a flavor enhancing agent for use in drinks which is capable of selectively enhancing the flavor of a flavoring of interest. Another object of the present invention is to provide a drink that contains the agent and which is enhanced in flavor.

### Solution to Problems

The present inventors conducted an intensive study with a view to solving the stated problems; as a result, they found that a polymerized catechin obtained from a tea extract was capable of selectively enhancing the flavor of a flavoring, in particular, the fruity and refreshing sensations of a fruit flavoring, without affecting the flavor of foods and drinks that contain it. They also found that when a polymerized catechin was added to a drink (in particular, low Brix drink), body and/or volume sensation were imparted and/or enhanced without impairing the flavor of the drink itself and, furthermore, that the flavor of a flavoring added (in particular, a fruit flavoring or a plant flavoring (excluding a tea-based flavoring)) could be enhanced selectively. The present invention has been accomplished on the basis of these findings.

Thus, the present invention relates to the following.

1. A flavor enhancing agent comprising a polymerized catechin as an active ingredient.
2. The flavor enhancing agent as recited in 1 which is an additive for use in a drink.
3. The flavor enhancing agent as recited in 2, wherein the drink is a tea-based beverage.
4. The flavor enhancing agent as recited in any one of 1 to 3, which is an agent for enhancing a fruit flavor.
5. A flavoring composition comprising a flavoring and a polymerized catechin.
6. The flavoring composition as recited in 5, wherein the concentration of the polymerized catechin is at least 0.0050 wt % of the total composition.
7. The flavoring composition as recited in 5 or 6, wherein the polymerized catechin is derived from a tea leaf extract.
8. The flavoring composition as recited in any one of 5 to 7, which further contains a non-polymerized catechin and wherein (a)/(b), or the ratio on a weight basis between (a) the contained polymerized catechin and (b) the contained non-polymerized catechin, is at least one.
9. The flavoring composition as recited in any one of 5 to 8, wherein the flavoring is a fruit flavoring.
10. A non-tea-based beverage which is a drink containing a polymerized catechin and a flavoring and wherein the concentration of the polymerized catechin is at least 0.0050 wt % of the total drink.
11. A tea-based beverage which is a drink containing a polymerized catechin and a flavoring and wherein the concentration of the polymerized catechin is at least 0.010 wt % of the total drink.
12. The beverage as recited in 10 or 11, wherein the polymerized catechin is derived from a tea leaf extract.
13. The beverage as recited in any one of 10 to 12, which further contains a non-polymerized catechin and wherein (a)/(b), or the ratio on a weight basis between (a) the contained polymerized catechin and (b) the contained non-polymerized catechin, is at least one.
14. The beverage as recited in any one of 10 to 13, wherein the flavoring is a fruit flavoring or a plant flavoring (excluding a tea-based flavoring).
15. The beverage as recited in any one of 10 to 14, which is a neutral drink (a drink with pH of 4.7-7.0).
16. The beverage as recited in any one of 10 to 15, which has a Brix value of 1.0 or less.
17. The beverage as recited in any one of 10 to 16, which is a drink free of a sweetener and/or an acidulant.
18. The beverage as recited in any one of 10 to 17, which is a tea-based beverage.
19. Use of a polymerized catechin for production of a flavor enhancing agent or for flavor enhancement.

### Advantageous Effect of Invention

When the flavor enhancing agent of the present invention is added to a drink, it does not affect the flavor of the drink per se but it can be used as an enhancer of the flavor of a flavoring incorporated in the drink.

The flavor enhancing agent of the present invention particularly serves as an enhancer of a fruit flavoring to enhance its fruity and refreshing sensations, so it can selectively enhance the fruit flavor of drinks such as a neutral tea-based beverage containing a fruit flavoring added thereto.

The drink of the present invention which comprises a polymerized catechin and a flavoring is a beverage that is provided with and/or enhanced in body and/or volume sensation without impairing the flavor of the beverage itself and which is also enhanced in the top and last notes of the flavoring. Even if the beverage is in the form of a packaged drink which is manufactured via a sterilization treatment, it is sufficiently enhanced in flavor to gain high consumer's preference.

### MODES FOR CARRYING OUT THE INVENTION

The term “flavor enhancing agent” as used herein refers to an additive for enhancing the flavor of foods and drinks and, specifically, it means an additive for exhibiting at least one, preferably all, of the following actions: enhancing the fragrance sensed by nose (top note) immediately before the food or drink is ingested; enhancing the fragrance in the mouth that is sensed by mouth when the food or drink is being ingested (or sipped); and enhancing the fragrance sensed in the aftertaste (last note).

(Polymerized Catechin)

The flavor enhancing agent of the present invention comprises a polymerized catechin as an active ingredient. The term “polymerized catechin” as used herein refers to such a structure that a plurality of yet to be polymerized monomeric catechins ((+)-catechin, (−)-epicatechin, (+)-gallocatechin, (−)-epigallocatechin, (−)-catechin gallate, (−)-epicatechin gallate, (−)-gallocatechin gallate, and (−)-epigallocatechin gallate (these are herein sometimes designated as “non-polymerized catechins”)) are connected by a tea-derived enzyme, an enzyme, light, and the like. Specifically, the polymerized catechin is a component that peaks at the same elution time as theaflavin (a product of Kurita Research Center, referential elution time: 24 minutes) when analyzed by HPLC under the following conditions.
Column. TSK-gel ODS-80TsQA (4.6 mm in diameter×150 mm, TOSOH CORPORATION)
Mobile phase: A: water:acetonitrile:trifluoroacetic acid=900:100:0.5
   B: water: acetonitrile:trifluoroacetic acid=200:800:0.5
Flow rate: 1.0 ml/min
Column temperature: 40° C.
Gradient conditions:
   0% of liquid B from initiation of the analysis to 5 minutes after initiation
   8% of liquid B from 5 minutes to 11 minutes
   10% of liquid B from 11 minutes to 21 minutes
   100% of liquid B from 21 minutes to 22 minutes
   Keep 100% of liquid B from 22 minutes to 30 minutes
   0% of the liquid B from 30 minutes to 31 minutes
Detection: A 280 nm (data collection time was 30 minutes); the peak area was used for quantification.
Injection volume: 10 μL
Reference material: Oolong homobisflavan B (abbreviated as OHBF-B)

The amount of polymerized catechin can be determined by constructing a calibration curve using OHBF-B as a reference material. Examples of the OHBF-B as a reference material include one that is synthesized by the method described in Chem. Pharm. Bull 37 (12), 3255-3563 (1989) or by the method described in Example 3 in JP 2005-336117 A (preferably purified to 98%© or higher purity) and one that is isolated from tea leaves.

Specific examples include the polymerized catechin called by such trivial names as thearubigin, as well as polymerized catechins exemplified by: an epigallocatechin gallate dimer represented by the formula (1):

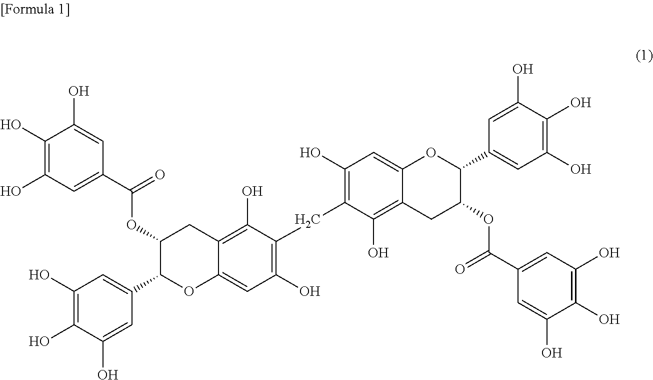

an epigallocatechin gallate trimer represented by the formula (2):

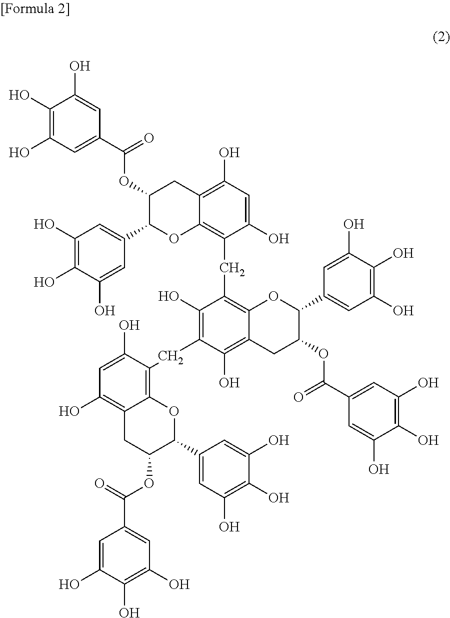

an epigallocatechin dimer represented by the formula (3):

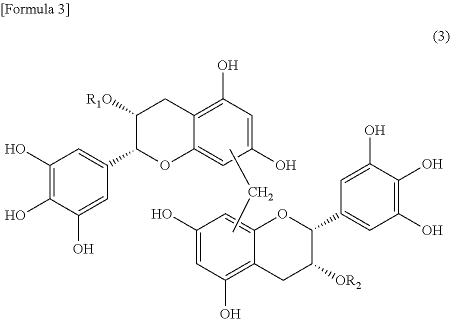

(wherein R₁ and R₂ each independently represent H or a galloyl group);
an epigallocatechin trimer represented by the formula (4):

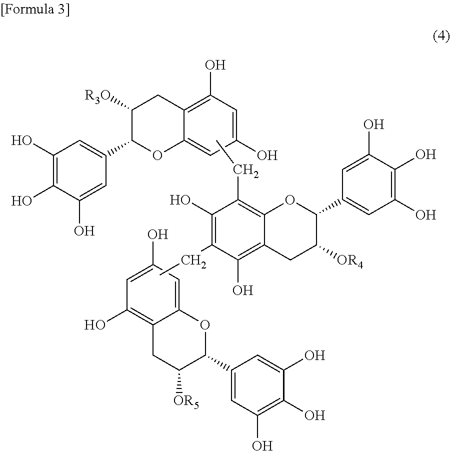

(wherein R₃, R₄ and R₅ each independently represent H or a galloyl group);
and an oolong theanine-3′-0-gallate represented by the formula (5):

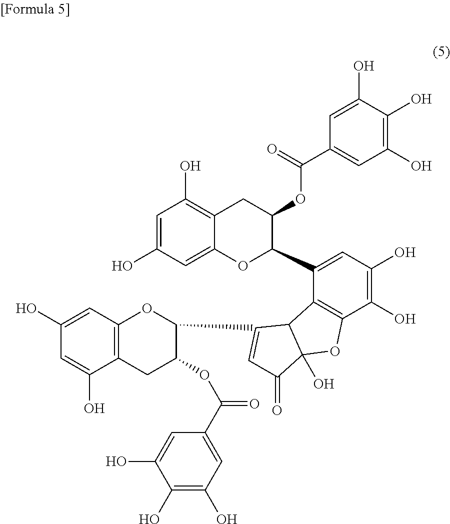

The polymerized catechin of the present invention is obtained as a plant extract containing polymerized catechins. Specifically, it can be obtained by subjecting tea leaves to extraction with a solvent. Tea leaves that can be used as the raw material may be of one, two or more kinds of tea selected from among green tea as unfermented tea, oolong tea as semi-fermented tea, and black tea as fermented tea. Among these, leaves of semi-unfermented tea or fermented tea which contain polymerized catechins in abundance are preferably used. Extraction solvents that can be used include cold or hot water, methanol, ethanol, isopropanol, ethyl acetate and the like, and extraction may be performed using these solvents either alone or in admixture of two or more kinds. The resulting solvent extract of tea leaves may be used as such, but it is preferably used in a concentrated or purified form that is obtained by selectively removing the components other than the polymerized catechin from the solvent extract of tea leaves to increase the content of the polymerized catechin.

Generally, non-polymerized catechins have a bitter and astringent taste, so if they are incorporated in large amounts in foods or drinks, the taste of the latter might be impaired. Hence, the polymerized catechin to be used in the present invention is preferably an extract with solvent that is obtained by a selective treatment for removing non-polymerized catechins from the above-mentioned tea leaf extract. The extract with solvent as obtained by the treatment for selectively removing the non-polymerized catechins may be exemplified by one that contains a polymerized catechin at a concentration four or more times greater than that of non-polymerized catechins, as described in WO2005/077384.

The polymerized catechin is a soluble component. Typically from the viewpoint of preventing precipitation during storage, the solvent extract of tea leaves is preferably subjected to a separation/clarification treatment such as centrifugal separation or filtration so as to remove insoluble solids; this is another reason that non-polymerized catechins are preferably removed to increase the concentration of the polymerized catechin in the solvent extract of tea leaves. The polymerized catechin to be used in the present invention may be in a liquid form or it may be reduced to a powder as by spray drying or frost shattering; if the food or drink is a beverage, it is preferred, from the viewpoint of solubility, to use a polymerized catechin after it is concentrated to a liquid form (or alternatively, it may be isolated or purified in a liquid form).

(Flavor Enhancing Agent)

The flavor enhancing agent of the present invention comprises a polymerized catechin as an active ingredient and when it is added to a food or drink, it enhances the flavor of a flavoring contained in the food or drink but without affecting the flavor of the food or drink itself; hence, the agent is useful as a flavor enhancer. The food or drink to which the flavor enhancing agent of the present invention can be added is not limited but it is preferably used as an additive for drinks. Generally, if the food or drink is in a liquid form (beverage), it is more susceptible to the taste of additives than when it is a solid preparation; the flavor enhancing agent of the present invention, because of its advantage of causing a smaller effect on the flavor of the food or drink per se, is particularly useful in drinks.

In addition, the polymerized catechin is less susceptible to heat and less likely to disappear during prolonged storage, so foods and drinks to which the flavor enhancing agent of the present invention has been added are characterized in that they are less likely to undergo changes over time. Although the underlying mechanism is not clear, the polymerized catechin would trap or surround the flavoring component in the food or drink to thereby prevent its dissipation.

The flavor enhancing agent of the present invention is also characterized in that it selectively enhances the flavor of a flavoring added to a food or drink. For example, if the food or drink is a tea beverage to which a flavoring has been added, the flavor enhancing agent of the present invention enhances the flavor of this flavoring to ensure that the top note, the fragrance in the mouth, and the last note will become rich. This effect is independent of the type of flavoring used but if the food or drink is a tea beverage, the flavor enhancing action is pronounced in the case where a non-tea-based flavoring (e.g., a fruit flavoring or a plant flavoring excluding a tea-based flavoring) is added to the beverage. The term “fruit flavoring” as used above refers to flavorings that present the flavor of fruit and include natural flavorings obtained from the edible reproductive portion of seed plants, in particular, the portion that accompanies the seed and has sweet pulp, as well as flavorings synthesized to produce fruit flavorings obtained from natural sources. Particularly preferred flavorings include citrus flavorings (e.g. orange, mandarin orange, grapefruit, lemon, lime, and bergamot), apple, grape, peach, tropical fruits (pineapple, guava, banana, mango, acerola, papaya, passion fruit, and coconut), and other fruits (e.g. plum, pear, apricot, prune, berry, cherry, strawberry, kiwi fruit, and melon); citrus flavorings are particularly preferred. These fruit flavorings may be obtained from natural sources such as fruit juice and fragrant oils, or they may be synthesized. If desired, fruit juice itself may be used as a flavoring component.

The term “plant flavoring” as used herein refers to flavorings that are obtained from portions of plants other than the fruit flavorings mentioned above and which are not tea-based flavorings (flavorings that present the flavor of tea leaves such as those of green tea, oolong tea, and black tea). Examples include flavorings obtained from nuts, tree bark, roots and leaves; also included are plant flavorings obtained from natural sources and flavorings synthesized to produce fruit flavorings obtained from natural sources. Such plant flavorings are exemplified by cola, mint, jasmine, rose, and coffee flavorings. These plant flavorings may be obtained from natural sources such as essence oils and essences or they may be synthesized. In the case of coffee, herbs such as mint or flowers such as jasmine, their essences (those obtained by subjecting plants to extraction with a solvent, optionally concentrated afterward) may be used as flavoring components.

The flavoring components may be used either alone or in admixture. The mixed flavoring may be exemplified by a citrus flavoring consisting of lime and lemon in admixture, the combination of a cola flavoring and a citrus flavoring, and the combination of pineapple and orange.

The flavor enhancing agent of the present invention also exhibits a marked action for enhancing a fruit flavor. It offers an additional advantage for foods and drinks having a fruit flavor: it is capable of not only enhancing or maintaining the fruity sensation but also imparting the characteristic refreshing sensation of fruit. Thus, the flavor enhancing agent of the present invention is particularly effective as an agent for enhancing a fruit flavor. In this connection, foods and drinks having a fruit flavor may be exemplified by foods and drinks that contain a fruit-derived component such as juice or pulp, as well as foods and drinks to which a fruit flavoring or the like has been added to impart a desired fruit flavor.

The form of a food or drink to which the flavor enhancing agent of the present invention is to be added is not particularly limited but since its effect is markedly exhibited when it is added to a drink, an advantageous mode is where the food or drink is in the form of a beverage. Considering that the flavor enhancing agent of the present invention is useful in enhancing a fruit flavor, preferred drinks are those having a fruit flavor which specifically include drinks in which fruit juice or pulp is incorporated, such as drinks containing fruit juice, refreshing drinks using fruit juice, drinks containing fruit pulp, and drinks containing juice sacks, as well as beverages that do not contain fruit juice or pulp (with no fruit juice or pulp added) but which are flavored with only a fruit flavoring (as in water, sports drinks, and tea-based beverages); particularly preferred are beverages in which the flavor improving effect will readily develop, which contain diluted fruit juice or have no fruit juice added, and which are lacking in body, fruit juice sensation, refreshing sensation, fully-ripened flavor, etc., in particular, beverages that are neutral (pH 4.7-7.0) and of low Brix value (no more than 1.0) and to which neither acidulant nor sweetener has been added.

If the flavor enhancing agent of the present invention is to be used as an additive for drinks, the amount of its addition to a drink may be chosen as appropriate for the composition of the drink and the consumer's preferene required of the drink; usually, the polymerized catechin or a plant extract (preferably a tea leaf extract) containing the polymerized catechin is added in such an amount that the polymerized catechin as an active ingredient approximately accounts for at least 0.005 wt % but less than 0.05 wt %, preferably at least 0.01 wt % but less than 0.045 wt %, and more preferably at least 0.02 wt % but less than 0.04 wt %, of the total amount of the drink. If the proportion of the polymerized catechin incorporated in the drink is less than 0.005 wt %, there may be a case where the flavor enhancing action is not fully exhibited.

As long as the polymerized catechin or a plant extract (preferably a tea leaf extract) containing the polymerized catechin is contained as an active ingredient in the flavor enhancing agent of the present invention, various other additives such as a dispersant, an excipient and a sweetener may also be contained depending on the object. The dispersant, excipient and sweetener should be such that they can taken into the body without causing any problem and they may be exemplified by reduced paratinose, various saccharides, organic acids or organic acid salts, starch, dextrin, dextran, and powdered milk. The flavor enhancing agent of the present invention may contain a solvent or a dispersant, as exemplified by water and ethanol. The flavor enhancing agent of the present invention is not particularly limited in form and it may assume any shape such as a powder, granules, paste or liquid.

(Flavoring Composition)

The flavor enhancing agent of the present invention is added to a food or drink to enhance the flavor of a certain fragrant component in them but if it is used in combination with a flavoring, it can selectively enhance the flavor of the flavoring, so it may be applied to provide a flavoring composition having an enhanced flavor.

The proportion of the polymerized catechin in the flavoring composition of the present invention is at least 0.005 wt %, preferably at least 0.01 wt %, and more preferably at least 0.02 wt %. If the proportion of the polymerized catechin incorporated in the flavoring composition is less than 0.005 wt %, there may be a case where the flavor enhancing action is not fully exhibited. There is no upper limit for the polymerized catechin in the flavoring composition and may be set as appropriate for the type of the flavoring, the amount in which it is incorporated, and the flavor to be enhanced; if the polymerized catechin is present in excess of the flavoring, the flavor of the polymerized catechin per se or a plant extract per se that contains the polymerized catechin may inhibit the flavor of the flavoring, so the proportions of the flavoring and the polymerized catechin in the flavoring composition of the present invention are such that the polymerized catechin approximately accounts for 0.05-5.0 parts by mass, preferably 0.06-2.0 parts by mass, more preferably 0.1-2.0 parts by mass, per part by mass of the flavoring.

As in the case of the already described flavor enhancing agent, the polymerized catechin may be replaced by either a plant extract (preferably a tea leaf extract) containing the polymerized catechin or a purified product thereof. If the polymerized catechin is to be incorporated as a tea leaf extract, the non-polymerized catechins contained in the tea leaf extract might inhibit the flavor of the flavoring (in particular, a fruit flavoring or a plant flavoring (excluding a tea-based flavoring)), so it is preferred to use the tea leaf extract after the non-polymerized catechins have been selectively removed. If the flavoring composition of the present invention is in the form of a drink which is a preferred mode as will be described later, the upper limit for the non-polymerized catechins in the drink is 0.025 wt % or less, preferably 0.02 wt % or less, more preferably 0.018 wt % or less, and even more preferably 0.015 wt % or less; further, (a)/(b), or the ratio on a weight basis between (a) the polymerized catechin and (b) non-polymerized catechin, is preferably at least one, more preferably at least 1.3, and even more preferably at least 1.6; from the viewpoint of the flavor enhancing action of the polymerized catechin, the upper limit of (a)/(b) is 3.0 or less, preferably 2.5 or less. If the drink is a non-tea-based beverage, it is preferably adjusted to contain none or only a minimum amount of non-polymerized catechins; if the drink is a tea-based beverage, non-polymerized catechins are an important factor that determines the taste of the tea-based beverage, so the non-polymerized catechins are preferably contained in a specified amount. If the drink is a tea-based beverage, the proportion of (a) the polymerized catechin to be incorporated in the drink approximately accounts for 0.01-0.05 wt %, preferably 0.01-0.03 wt %, and more preferably 0.02-0.03 wt %, of the total amount of the drink whereas the proportion of (b) the non-polymerized catechins to be incorporated in the drink accounts for 0.002-0.025 wt %, preferably 0.01-0.02 wt %, and more preferably 0.014-0.015 wt %, of the total amount of the drink, and (a)/(b), or the ratio on a weight basis between (a) the polymerized catechin and (b) non-polymerized catechin, is preferably at least one, more preferably at least 1.3, and even more preferably at least 1.6, with the upper limit of (a)/(b) being 3.0 or less, preferably 2.5 or less.

The flavoring composition of the present invention is less susceptible to heat and even if it is produced via a heat sterilization treatment such as UHT sterilization or retort sterilization, the possible decrease in flavor is suppressed; it is, therefore, used with advantage in foods and drinks that are produced via a sterilization treatment, for example, packaged drinks.

If the flavoring composition of the present invention is used, body and volume sensation are imparted or enhanced and if the flavoring is a fruit flavoring, a plant flavoring or a mixture thereof, it provides the flavor of the flavoring with volume sensation, juicy sensation, depth sensation, mild sensation, refreshing sensation, and fully-ripened sensation, thereby offering the advantage of creating a flavor that is close to natural fruits or plants. The fruit flavoring or plant flavoring may be of the same kinds as already described for the flavor enhancing agent.

In the present invention, a mixture of the polymerized catechin and a flavoring may be incorporated in a food or drink as an additive; alternatively, the polymerized catechin and a flavoring may be added separately in a food or drink to provide a form of food or drink having an enhanced flavor. As already noted above, the flavor enhancing action of the present invention is readily exhibited in drinks, so drinks are the most preferred form as a mode for the composition of the present invention. If the flavoring composition of the present invention is in the form of a drink, the proportion of the polymerized catechin in the composition approximately accounts for 0.005-0.05 wt %, preferably 0.01-0.03 wt %, and more preferably 0.02-0.03 wt %, whereas it is usual for the proportion of the flavoring to approximately account for 0.001-1.0 wt %, preferably 0.005-0.50 wt %, and more preferably 0.01-0.30 wt %. If the drink is a tea-based beverage, it is preferred to utilize the ranges previously mentioned.

It is preferred that the flavoring composition of the present invention further contains caffeine. According to the study of the present inventors, caffeine incorporated in a specified amount will synergistically increase the flavor enhancing action of the polymerized catechin. If the flavoring composition of the present invention is in the form of a drink, caffeine is incorporated in an approximate amount of 0.005-0.03 wt %, preferably 0.01-0.025 wt %, and more preferably 0.01-0.02 wt %.

The flavoring composition of the present invention will readily exhibit its flavor enhancing effect in foods and drinks having a fruit flavor or a plant flavor (excluding a tea-based flavor), particularly in drinks containing diluted fruit juice, low Brix drinks having a fruit flavoring or a plant flavoring added thereto, or neutral drinks to which a fruit flavoring or a plant flavoring has been added but neither an acidulant nor a sweetener has been added. The composition tends to be most effective in neutral drinks to which neither an acidulant nor a sweetener has been added.

Specifically, drinks containing diluted fruit juice are beverages that contain fruit juice in an amount of 50% or less, preferably 30% or less, more preferably 20% or less, even more preferably 10% or less, and most preferably 5% or less (even 0% fruit juice is contemplated), and examples include refreshing beverages such as near waters, sports drinks with the fruit juice content being about 1%. The “fruit juice” as referred to herein shall include plant extracts (essences) for the sake of convenience. Drinks containing diluted fruit juice are usually lacking body, juicy sensation, refreshing sensation, fully-ripened sensation, etc. but those drinks incorporating the polymerized catechin at a specified concentration according to the present invention are provided with body and volume sensation, as well as juicy sensation, depth sensation, mild sensation, refreshing sensation, and fully-ripened sensation, thereby creating a flavor that is close to natural fruits or plants. Speaking of drinks with a fruit juice content of 50% or less, the percentage represents a relative concentration of fruit juice, with the concentration of straight juice squeezed from a fruit being taken as 100%; this is a value calculated on the basis of either the reference for readings on a sugar refractometer (° Brix) or the reference for acidity (%), both specified in the JAS standards (the Japanese Agricultural Standards for fruit drinks).

Specifically, low Brix drinks are beverages with a Brix value of 1.0 or less, preferably 0.8 or less, more preferably 0.6 or less, and even more preferably 0.5 or less, that are lacking in body and volume sensation, as well as flavor (top note and fragrance in the mouth), or which have a lingering aftertaste but are lacking in the last note on account of the use of a high-intensity sweetener. Take, for example, cola beverages with a Brix value of about 0.5 (diet cola beverages using a high-intensity sweetener); they can be flavored only less effectively with flavorings than cola beverages with a Brix value of about 10 that are prepared from a recipe incorporating saccharides (e.g. sucrose) and they are also lacking in the spicy sensation and citrus sensation which are characteristic of cola. In contrast, the drink of the present invention that contains a specified concentration of the polymerized catechin and a cola flavoring is provided with body sensation and volume sensation that are comparable to those exhibited by the cola beverages with a Brix value of about 10.0, whereby the drink is enhanced in the flavor of spicy sensation and citrus sensation and is yet improved with respect to the aftertaste of the high-intensity sweetener.

Specifically, drinks to which neither an acidulant nor a sweetener has been added are beverages with neutrality (pH 4.7-7.0, preferably pH 5.0-7.0, and more preferably pH 5.5-6.5) and a Brix value of 1.0 or less (preferably 0.8 or less, more preferably 0.6 or less, and even more preferably 0.5 or less), and examples include water, tea-based beverages and others that have the flavor of a fruit flavoring or a plant flavoring. Drinks to which neither an acidulant nor a sweetener has been added are such that even if a flavoring is incorporated, its flavor is barely perceived (poor top note, inadequate fragrance in the mouth, and negligible last note) and, in particular, fruit flavorings or plant flavorings (excluding tea-based flavorings) are less effective in developing the intended fruit flavor or plant flavor in the absence of an acidulant or a sweetener; in contrast, the drink of the present invention that contains a specified concentration of the polymerized catechin and the flavoring has the advantage that even if neither an acidulant nor a sweetener has been added to it or even if neither fruit juice nor a plant extract (essence) has been added, it has a rich top note that is characteristic of a fruit or plant, maintains a refreshing fragrance in the mouth, and gives an aftertaste that allows the consumer to enjoy the fragrance of the fruit or plant (last note). Particularly in the case of drinks that are tea-based beverages, components such as non-polymerized catechins which are derived from tea leaves interfere with effective impartment of the flavor of a flavoring and if a fruit flavoring or a plant flavoring is added, the consumer can hardly sense their flavor. However, the drink of the present invention that contains a specified concentration of the polymerized catechin and a fruit flavoring or a plant flavoring provides a drink that allows the consumer to enjoy the fragrance of the fruit flavoring or the plant flavoring simultaneously with the fragrance of the tea-based beverage that serves as the base of the drink.

The term “tea-based beverage” as used herein represents tea-based beverages that have incorporated therein a tea leaf extract obtained by a process in which tea leaves obtained by processing the tea leaves of the genus Camellia, such as C. sinensis, C. assamica, yabukita species, and hybrids thereof, are subjected to extraction with cold water, hot water or an aqueous solution having an extraction aid added thereto. Processed tea leaves include those of unfermented teas such as green tea (e.g. sencha, bancha, gyokuro, tencha, and kamairi-cha), semi-fermented teas such as oolong tea (e.g. tetsukannon, irotane, ougonkei, and buigancha), and fermented teas such as black tea (e.g. Darjeeling, Assam, and Sri Lanka); the tea leaf extract to be incorporated in the tea-based beverage of the present invention may be obtained by subjecting any of these tea leaves to extraction. Note that beverages that do not belong to the above-defined tea-based beverage are herein designated as “non-tea-based beverages.”

The term “acidulant” as used herein refers to a component that presents an acidic taste and examples include fruit juices extracted from natural ingredients, as well as fumaric acid, phosphoric acid, citric acid, lactic acid, etc. The term “sweetener” refers to a component that presents a sweet taste. Examples include sucrose, isomerized sugar, glucose, fructose, lactose, maltose, xylose, isomerized lactose, fructooligosaccharide, maltooligosaccharide, isomaltooligosaccharide, galactooligosaccharide, coupling sugar, paratinose, maltitol, sorbitol, erythritol, xylitol, lactitol, paratinit, saccharification product of reduced starch, stevia, glycyrrhizin, thaumatin, monelin, aspartame, alitame, saccharin, acesulfame-K, sucralose, dulcin, neotame, etc.

If the mixture of the polymerized catechin and a flavoring is to be incorporated as an additive in a food or drink, the flavoring composition of the present invention, as long as it contains the two active ingredients, i.e., the flavoring and the polymerized catechin or a plant extract (preferably a tea leaf extract) containing the polymerized catechin, may contain various other additives such as a dispersant, an excipient and a sweetener, depending on the object. The dispersant, excipient and sweetener should be such that they can be taken into the body without causing any problem and they may be exemplified by reduced paratinose, various saccharides, organic acids or organic acid salts, starch, dextrin, dextran, and powdered milk. The flavoring composition of the present invention may contain a solvent or a dispersant, which may be exemplified by water, ethanol, etc. The flavoring composition of the present invention is not particularly limited in form and it may take on any form such as a powder, granules, paste, liquid, etc.

If the polymerized catechin and a flavoring are to be added separately to a food or drink, preferably in the case where the flavoring composition of the present invention is in the form of a drink, the drink of the present invention may have various additives incorporated therein, either independently or in combination; exemplary additives include an antioxidant, a flavoring, inorganic acids, inorganic acid salts, inorganic salts, pigments, an emulsifier, a preservative, a seasoning (flavoring), a sweetener, an acidulant, fruit juice, a pH modifier, and a quality stabilizer.

As noted above, the flavoring composition of the present invention is less susceptible to heat and even if it is produced via a heat sterilization treatment such as UHT sterilization or retort sterilization, the possible decrease in flavor is suppressed; it may, therefore, be used with advantage in packaged drinks. The package in which the composition can be supplied on the market may assume any form, such as a polylethylene terephthalate based, shaped container (so-called PET bottle), a metal can, a paper container that consists of metal foil combined with plastic film, or a bottle. The drinks referred to above shall cover two types for the sake of convenience, one that can be drunk without being diluted, and the other being so-called “syrup” which is to be diluted before being drunk.

The packaged drink of the present invention can be manufactured by any known methods; in the case where heat sterilization can be performed on a container such as a metal can after it has been filled with a beverage, the packaged drink is manufactured by retort sterilization or other suitable methods under the sterilizing conditions specified in the Food Sanitation Act. In the case of PET bottles, paper containers and others that cannot be sterilized by retorting, a plate-type heat exchanger or other device may be used to sterilize the beverage at high temperature for a short time and, thereafter, the beverage is cooled to a specified temperature and packed into the container. If desired, after this packing operation, other components may be incorporated in the packed container under aseptic conditions.

### EXAMPLES

The present invention is described below in greater detail by reference to Examples. However, the present invention is not limited by these Examples.

### Production Example 1

### Production of an Essence Having High Content of Polymerized Catechin

Using 7800 kg of a sodium bicarbonate solution which consisted of 0.15 wt % sodium bicarbonate added to hot water (95° C.), 600 kg of oolong tea leaves was subjected to extraction, yielding a liquid oolong tea extract in an approximate amount of 7000 kg. With its temperature held at 60-65° C., the liquid extract was flowed through 400 kg of particulate activated carbon (GW-H32/60; product of KURARAY CO., LTD.) to remove non-polymerized catechins and caffeine. The resulting effluent (from the treatment with activated carbon) was concentrated under reduced pressure to give approximately 900 kg of an essence with a Brix value of 11 having high content of polymerized catechin (essence as a concentrated oolong tea; hereinafter called essence A). The concentrations of polymerized catechins, non-polymerized catechins and caffeine in essence A thus obtained were measured by HPLC under the following conditions. As a result, the polymerized catechins, non-polymerized catechins and caffeine were found to be present in respective amounts of 12000 ppm, 800 ppm and 20 ppm on a weight basis.

HPLC Conditions:
Column: TSK-gel ODS-80TsQA (4.6 mm in diameter×150 mm, TOSOH CORPORATION)
Mobile phase: A: water:acetonitrile:trifluoroacetic acid=900:100:0.5
   B: water:acetonitrile:trifluoroacetic acid=200:800:0.5
Flow rate: 1.0 ml/min
Column temperature: 40° C.
Gradient conditions:
   0% of liquid B from initiation of the analysis to 5 minutes after initiation
   8% of liquid B from 5 minutes to 11 minutes
   10% of liquid B from 11 minutes to 21 minutes
   100% of liquid B from 21 minutes to 22 minutes
   Keep 100% of liquid B from 22 minutes to 30 minutes
   0% of liquid B from 30 minutes to 31 minutes
Detection: A 280 nm
Reference material: Oolong homobisflavan B (OHBF-B)
Retention time of the polymerized catechin: A peak observed at approximately 25 minutes coincided with the peak of theaflavin.

### Production Example 2

### Production of Polymerized Catechin (as Purified Product)

Using a sodium bicarbonate solution which consisted of 0.15 wt % sodium bicarbonate added to hot water (95° C.), oolong tea leaves were subjected to extraction, yielding a liquid oolong tea extract. The liquid extract was freeze-dried and 8 g of the dried product was subjected to preparative chromatography under the following conditions. Components that eluted in 59.8-70 minutes were combined and freeze-dried to yield a polymerized catechin (as purified product).

HPLC conditions:
Column. ODS-10/20 (50*300 nm+50*100 mm; product of NOMURA CHEMICAL CO., LTD.)
Mobile phase: A: water:acetonitrile:trifluoroacetic acid=900:100:0.5
   B: water:acetonitrile:trifluoroacetic acid=200:800:0.5
Flow rate: 60 ml/min
Column temperature: 40° C.
Gradient conditions:
   0% of liquid B from initiation of the analysis to 52 minutes after initiation
   100% of liquid A from 52 minutes to 52.1 minutes
   100% of liquid A from 52.1 minutes to 79 minutes
   100% of liquid B from 79 minutes to 81 minutes
   Keep 100% of liquid B from 81 minutes to 100 minutes
Detection: A 280 nm
Injection volume: 15 ml

### Example 1

### Flavor Enhancing Action of Polymerized Catechin (Essence A)

To ensure that the polymerized catechin content would range from 0 to 500 ppm (0-0.05 wt %) as indicated in Table 1, water was added to the concentrate of tea extract as obtained in Production Example 1 (polymerized catechin containing essence, or essence A). To the solutions thus obtained, a lemon flavoring was added in an amount of 0.15 wt % and the mixtures were sterilized under the condition of 85° C.×10 min and then cooled to 5° C. to prepare samples of lemon-flavored water (pH 6.2-6.5; Brix 0.01-0.46). These samples were put to a sensory test for evaluation by three professional panelists. The criterion of the sensory test was the flavor enhancing action, or the intensity of lemon flavor, as compared with a control to which the polymerized catechin (essence A) had not been added, and an overall evaluation was made for the enhancement of fragrance before sipping (top note), the enhancement of fragrance upon sipping, and the enhancement of fragrance in the aftertaste (last note), on the basis of the following 5-score rating: 1, the fruit flavor not weak as compared with the control; 2, no difference; 3, somewhat strong; 4, strong; 5, considerably strong.

The results are shown in Table 1. Upon addition of the polymerized catechin, the lemon flavor was enhanced in all aspects, top note, fragrance in the mouth, and last note. The enhanced lemon flavor had refreshing sensation and fully-ripened sensation and was more like natural lemon fruit than the control was. This suggested that the polymerized catechin had a flavor enhancing action (fruit flavor enhancing action). It should, however, be noted that when the concentration of the polymerized catechin was 500 ppm (0.05 wt %) or more, one panelist had the impression that the flavor presented by essence A inhibited the lemon flavor. These observations suggested that an optimum concentration of the polymerized catechin ranged from 50 ppm to less than 500 ppm (from 0.0050 wt % to less than 0.05 wt %) and that by using the polymerized catechin in this concentration range with a flavoring, the intended flavor was enhanced to present a rich flavorl even in the sterilized drink.

TABLE 1

| | | | | | | | | |
|---|---|---|---|---|---|---|---|---|
| Polymerized | 0 | 50 | 100 | 200 | 250 | 275 | 300 | 500 |
| catechin (ppm) | (con- | | | | | | | |
| | trol) | | | | | | | |
| Non-polymerized | 0 | 3 | 7 | 13 | 17 | 18 | 20 | 33 |
| catechin (ppm) | | | | | | | | |
| Sensory score | 2 | 3 | 4 | 5 | 4 | 3.5 | 2.5 | 1 |

### Example 2

### Flavor Enhancing Action of Polymerized Catechin

Samples of lemon-flavored water were prepared as in Example 1, except that essence A of Example 1 was replaced by the polymerized catechin (as purified product) prepared in Production Example 2. To be more specific, the polymerized catechin was added to water to give concentrations of 0-500 ppm; to the solutions thus obtained, a lemon flavoring was added in an amount of 0.15 wt % and the mixtures were sterilized under the condition of 85° C.×10 min and then cooled to 5° C. to prepare samples of lemon-flavored water (pH 6.0-6.5; Brix 0.01-0.05). These samples were put to a sensory test for evaluation by three professional panelists. The criterion of the sensory test was the flavor enhancing action, or the intensity of lemon flavor, as compared with a control to which the polymerized catechin had not been added, and an overall evaluation was made for the enhancement of fragrance before sipping (top note), the enhancement of fragrance upon sipping, and the enhancement of fragrance in the aftertaste (last note), on the basis of the following 5-score rating: 1, the fruit flavor not weak as compared with the control; 2, no difference; 3, somewhat strong; 4, strong; 5, considerably strong.

The results are shown in Table 2. As in Example 1, upon addition of the polymerized catechin, the lemon flavor was enhanced in all aspects, top note, fragrance in the mouth, and last note. Compared to Example 1 in which essence A (concentrated tea extract) was used, the purified product of polymerized catechin had a smaller effect on the flavor when it was added at high concentration (say, 500 ppm of the polymerized catechin). This observation suggested that when a plant extract containing the polymerized catechin is to be used, the degree of purification of the polymerized catechin in it should be increased.

TABLE 2

| | Polymerized catechin (ppm) | | | | |
|---|---|---|---|---|---|
| | 0 (control) | 100 | 200 | 300 | 500 |
| Non-polymerized | 0 | 0 | 0 | 0 | 0 |
| catechin | | | | | |
| (ppm) | | | | | |
| Sensory score | 2 | 4 | 4 | 3.5 | 3 |

### Example 3

### Flavor Enhancing Effect (1) of Polymerized Catechin in Tea Drink

Oolong tea leaves were subjected to extraction with 30 volumes of hot water for 4 minutes to prepare a liquid oolong tea extract. To the resulting liquid oolong tea extract (non-polymerized catechins, 145 ppm; polymerized catechins, 80 ppm), essence A prepared in Production Example 1 was added to prepare samples of oolong tea drink containing the polymerized catechin at concentrations of 500, 300, 280, 260, and 200 ppm; to the samples thus obtained, a lemon flavoring was added in an amount of 0.15 wt % and the mixtures were heat sterilized and then cooled to prepare samples of lemon-flavored oolong tea drink (pH 5.9; Brix 0.3-0.5). These samples were put to a sensory test by five professional panelists; the criterion of the sensory test was the quality of the flavor of each sample as flavored tea, namely, the balance between the flavor of oolong tea as the base and the lemon flavor of each sample, as compared with a control to which essence A had not been added, and evaluation was made on the basis of the following 5-score rating: 5, delicious, both oolong tea and lemon flavor could be enjoyed [symbol] 1, not so delicious, poor balance, with either oolong tea or lemon flavor being sticking out.

The results are shown in Table 3. Oolong tea drinks usually contain non-polymerized catechins at higher concentrations than the polymerized catechins (as shown by the control) but the samples that had the polymerized catechins added in accordance with the present invention were drinks that contained the polymerized catechin at higher concentrations than non-polymerized catechins; specifically, they were tea-based beverage samples wherein (a)/(b), or the ratio on a weight basis between (a) the polymerized catechin and (b) non-polymerized catechins, was at least one. By increasing the amount of the polymerized catechin, the body and volume sensation of the oolong tea drink were enhanced and the lemon flavor was found to have been selectively enhanced. This lemon flavor had juicy sensation, depth sensation, mild sensation, refreshing sensation, and fully-ripened sensation and was close to natural lemon fruit. The oolong tea drink having the lemon flavor enhanced by the polymerized catechin was a tea-based beverage with a fruit flavor that had sufficiently high drinkability (consumer's preference) to allow the consumer to enjoy both the taste of the base oolong tea itself and the lemon flavor.

TABLE 3

| | | | | | | |
|---|---|---|---|---|---|---|
| Polymerized | 80 | 200 | 260 | 280 | 300 | 500 |
| catechin (a) | (con- | | | | | |
| (ppm) | trol) | | | | | |
| Non- | 145 | 153 | 157 | 160 | 165 | 173 |
| polymerized | | | | | | |
| catechin (b) | | | | | | |
| (ppm) | | | | | | |
| Ratio (a/b) | 0.55 | 1.31 | 1.66 | 1.75 | 1.81 | 2.89 |
| Sensory score | 1.3 | 3.3 | 4.0 | 3.7 | 3.3 | 1.7 |

### Example 4

### Flavor Enhancing Effect (2) of Polymerized Catechin in Tea Drink

Using essence A prepared in Production Example 1 and the liquid oolong tea extracts prepared in Example 3, six samples of oolong tea drink were prepared; they had different ratios between non-polymerized catechins and polymerized catechins as shown in Table 4. To the samples thus obtained, a lemon flavoring was added in an amount of 0.15 wt % and the mixtures were heat sterilized and then cooled to prepare samples of lemon-flavored oolong tea drink (pH 5.9; Brix 0.3-0.5). These samples were put to a sensory test for evaluation by five professional panelists; the criterion of the sensory test was the quality of the flavor of each sample as flavored tea, namely, the balance between the flavor of oolong tea as the base and the lemon flavor of each sample, as compared with a control to which essence A had not been added, and evaluation was made on the basis of the following 5-score rating: 5, delicious, both oolong tea and lemon flavor could be enjoyed [symbol] 1, not so delicious, poor balance, with either oolong tea or lemon flavor sticking out.

The results are shown in Table 4. In tea-based beverages, non-polymerized catechins inhibit a fruit flavor but as it turned out, a satisfactory fruit flavor was presented by reducing the amount of non-polymerized catechins. As in Example 3, the samples wherein (a)/(b), or the ratio on a weight basis between (a) the polymerized catechins and (b) non-polymerized catechins, was at least 1, preferably at least 1.3, more preferably at least 1.7 were a beverage having high drinkability as flavored tea (i.e., having an enhanced lemon flavor and allowing the consumer to enjoy a good balance between the taste of oolong tea and the lemon flavor.) As one can see from Table 4, it was suggested that the concentration of non-polymerized catechins should preferably be 180 ppm or less, more preferably 150 ppm or less.

TABLE 4

| | | | | | | |
|---|---|---|---|---|---|---|
| Polymerized | 260 | 260 | 260 | 260 | 260 | 260 |
| catechin (a) | | | | | | |
| (ppm) | | | | | | |
| Non- | 10 | 20 | 110 | 145 | 180 | 270 |
| polymerized | | | | | | |
| catechin (b) | | | | | | |
| (ppm) | | | | | | |
| Ratio (a/b) | 26.0 | 13.0 | 2.36 | 1.79 | 1.44 | 0.96 |
| Sensory score | 3.3 | 3.5 | 4.0 | 4.2 | 3.0 | 1.8 |

### Example 5

### Flavor Enhancing Effect (3) of Polymerized Catechin in Tea Drink

Using essence A prepared in Production Example 1 and the liquid oolong tea extracts prepared in Example 3, three samples of oolong tea drink were prepared; they had different ratios between non-polymerized catechins and polymerized catechins as shown in Table 5. To each of the samples thus obtained, four flavorings, peach, bergamot, litchi, and peppermint, were added. The thus prepared samples were put to a sensory test for evaluation by five professional panelists; the criterion of the sensory test was the quality of the flavor of each sample as flavored tea, namely, the balance between the flavor of oolong tea as the base and the flavor of each flavoring, and evaluation was made on the basis of the following 5-score rating: 5, delicious, both oolong tea and the flavor of each flavoring could be enjoyed [symbol] 1, not so delicious, poor balance, with either oolong tea or the flavor of a flavoring sticking out.

The results are shown in Table 5. For each of the four flavorings, peach, bergamot, litchi and peppermint, the flavor enhancing effect of the polymerized catechin increased with increasing amount, improving the drinkability of all samples as flavored tea. In all samples, the flavors of the flavorings used were more enhanced by reducing the amount of non-polymerized catechins.

TABLE 5

| Flavor | Peach | | | Bergamot | | | Litchi | | | Peppermint | | |
|---|---|---|---|---|---|---|---|---|---|---|---|---|
| Polymerized | 260 | 260 | 260 | 260 | 260 | 260 | 260 | 260 | 260 | 260 | 260 | 260 |
| catechin (a) (ppm) | | | | | | | | | | | | |
| Non-polymerized | 120 | 150 | 180 | 120 | 150 | 180 | 120 | 150 | 180 | 120 | 150 | 180 |
| catechin (b) (ppm) | | | | | | | | | | | | |
| Ratio (a/b) | 2.17 | 1.73 | 1.44 | 2.17 | 1.73 | 1.44 | 2.17 | 1.73 | 1.44 | 2.17 | 1.73 | 1.44 |
| Sensory score | 5 | 4.6 | 3.6 | 4.6 | 4.4 | 4 | 4.4 | 4.2 | 3.4 | 4.8 | 4.6 | 3.4 |

### Example 6

### Cola-Flavored Drink

Essence A prepared in Production Example 1 was used as polymerized catechins. With aspartame (0.4 g) and acesulfame-K (0.1 g) being used as high-intensity sweeteners, citric acid (anhydrous) (0.55 g), trisodium citrate (0.5 g) and carbonated water (800 g) were mixed, and after mixing 0-200 ppm (0-0.02 wt %) of caffeine, 0.3 wt % of a cola flavoring, and essence A containing the polymerized catechins at concentrations of 0-400 ppm (0-0.04 wt %), water was added in a sufficient quantity to make a total volume of 1000 g, thereby preparing carbonated drink samples having a sweetness of 10, Brix values of about 0.16-0.47, and a gas pressure of 3.4 kg/cm². The thus prepared carbonated drink samples were put to a sensory test by four professional panelists to evaluate their flavor. To this end, sugar was incorporated in those samples in the amounts indicated in Table 6 to prepare test specimens having Brix values of 1, 2, 5 and 10 (sweetness: 10) and those specimens were subjected to a relative evaluation of body, volume sensation, and the intensity of the cola flavoring on the basis of the following rating system: 1, somewhat poor; 2, fairly perceptible; 3, perceptible; 4, highly perceptible.

The results are shown in Table 7. As the addition of the polymerized catechin increased, body sensation was imparted to the low Brix carbonated drink samples and the citrus sensation and spicy sensation of the cola flavoring were enhanced. When the polymerized catechin was added in amounts of 300 ppm and more, the low Brix carbonated drink samples acquired body sensation and flavor enhancement at comparable levels to the cola drink specimen having a Brix value of 5 or 10 which was prepared using sucrose. The carbonated drink samples containing 100 ppm or more of caffeine at the polymerized catechin concentration of 200 ppm, 50 ppm or more of caffeine at the polymerized catechin concentration of 300 ppm, and 50 ppm or more of caffeine at the polymerized catechin concentration of 400 ppm possessed comparable levels of body and volume sensation and flavor sensation to the carbonated drink specimen with a Brix value of 10 which was prepared using sucrose to gain high consumer's preference; thus, those carbonated drink samples were a beverage that had an extremely high degree of consumer's preference.

TABLE 6
(in grams)

| | Brix.1 | Brix.2 | Brix.5 | Brix.10 |
|---|---|---|---|---|
| Citric acid 3 Na | 0.50 | 0.50 | 0.50 | 0.50 |
| Citric acid | 0.55 | 0.55 | 0.55 | 0.55 |
| (anhydrous) | | | | |
| Aspartame | 0.36 | 0.32 | 0.20 | 0.0 |
| Acesulfame-K | 0.09 | 0.08 | 0.05 | 0.0 |
| Sucrose | 10 | 20 | 50 | 100 |
| Cola flavoring | 3 | 3 | 3 | 3 |
| Water | 200 | 200 | 200 | 200 |
| Carbonated water | 800 | 800 | 800 | 800 |
| Total | 1000 | 1000 | 1000 | 1000 |
| Rating score | 1 | 2 | 3 | 4 |

TABLE 7

| Polymerized catechin (ppm) | | | | | |
|---|---|---|---|---|---|
| (Lower, parenthesized figures | | | | | |
| represent the concentration of | Caffeine (ppm) | | | | |
| non-polymerized catechin) | 0 | 50 | 100 | 150 | 200 |
| 0 | 0 | 0.0 | 0.1 | 0.1 | 0.3 |
| (0) | | | | | |
| 100 | 1.0 | 1.5 | 1.9 | 2.1 | 1.8 |
| (7) | | | | | |
| 200 | 1.8 | 2.3 | 2.8 | 2.9 | 2.6 |
| (13) | | | | | |
| 300 | 2.5 | 3.1 | 3.5 | 3.8 | 3.5 |
| (20) | | | | | |
| 400 | 3.3 | 3.8 | 3.8 | 3.8 | 3.6 |
| (26) | | | | | |

1. A flavor enhancing agent comprising a polymerized catechin as an active ingredient.

2. The flavor enhancing agent according to claim 1 which is an additive for use in a drink.

3. The flavor enhancing agent according to claim 2, wherein the drink is a tea-based beverage.

4. The flavor enhancing agent according to claim 1, which is an agent for enhancing a fruit flavor.

5. A flavoring composition comprising a flavoring and a polymerized catechin.

6. The flavoring composition according to claim 5, wherein the concentration of the polymerized catechin is at least 0.0050 wt % of the total composition.

7. The flavoring composition according to claim 5, wherein the polymerized catechin is derived from a tea leaf extract.

8. The flavoring composition according to claim 5, which further contains a non-polymerized catechin and wherein (a)/(b), or the ratio on a weight basis between (a) the contained polymerized catechin and (b) the contained non-polymerized catechin, is at least one.

9. The flavoring composition according to claim 5, wherein the flavoring is a fruit flavoring.
